(19) United States
(12) Patent Application Publication
(10) Pub. No.: US 2023/0022094 A1
(43) Pub. Date: Jan. 26, 2023

(54) Use of Glucose Control Indicators for Risk Assessment and Treatment of Neurodevelopmental Disorders and Techniques for Establishing the Status of Chronic Glucose Control

(71) Applicant: Tammy Movsas, Southfield, MI (US)

(72) Inventor: Tammy Movsas, Southfield, MI (US)

(21) Appl. No.: 17/788,719

(22) PCT Filed: Nov. 25, 2020
(86) PCT No.: PCT/US20/62340

§ 371 Date: Jun. 23, 2022

Related U.S. Application Data
(60) Provisional application No. 62/958,029, filed on Jan. 7, 2020.

(51) Int. Cl. A61B 5/00; G01N 33/72; G01N 33/68; A61B 5/145

(52) U.S. Cl. CPC A61B 5/7275; G01N 33/723; G01N 33/6896; A61B 5/14532; A61B 2503/02; A61B 2503/045

(57) ABSTRACT

Dysglycemia as a risk factor for neurodevelopmental disorder or developmental diabetes. The risk is assessed based on measurement of a glucose control indicator in a blood sample. One particular example of a neurodevelopmental disorder is retinopathy of prematurity in an infant. One particular example of a glucose control indicator is ‘comprehensive glycated hemoglobin fraction’ or ‘comprehensive glycated albumin fraction.’ This is calculated using ‘total whole blood protein’ in the denominator. In the case of chronic hyperglycemia, there is an increased risk of proliferative retinopathy of prematurity. In the case of chronic hypoglycemia, there is an increased risk of non-proliferative retinopathy of prematurity. This ‘total whole blood protein’ technique could also be used to determine the glucose control status in other types of patients.

### TECHNICAL FIELD

The invention relates to medical conditions that result from abnormal glucose regulation and methods for assessing thereof, using biomarkers for establishing the status of chronic glucose control, for treatment planning and for monitoring medical conditions.

### BACKGROUND

Neurodevelopmental disorders are disorders which affect the development of the central nervous system, which includes the brain and the retina. Any child can be diagnosed with a neurodevelopmental disorder. However, children who were born prematurely are at higher risk of these disorders compared to children who were born full-term. For instance, cerebral palsy and autism are examples of neurodevelopmental disorders. Cerebral palsy and autism can occur in any child, but they more commonly occur in children who were born prematurely. Some neurodevelopmental disorders, such as retinopathy of prematurity, often referred to by its acronym, ROP, occur primarily in premature infants. ROP is especially prevalent in premature infants who were born before 30 weeks of gestational age.

The disruption of pathways leading to neurodevelopmental disorders often overlap with each other. This supports the scientific consensus that a common biological occurrence exists amongst them. This is the likely explanation why children are often diagnosed with more than one neurodevelopmental disorder. For example, it is not uncommon for cerebral palsy and autism spectrum disorder to occur together. Sometimes, neurodevelopmental disorders manifest during infancy, and other times, they do not manifest until later. Once again, cerebral palsy and autism spectrum disorder are examples of this. Sometimes, these diagnoses are made during infancy. But other times, these disorders are not diagnosed until 3-5 years age (even though the problem with neurodevelopment occurred prior to that). Some neurodevelopmental disorders are precursors of others, and thus an increased risk for one of these disorders predisposes the patient to additional neurodevelopmental disorders. For example, periventricular leukomalacia (PVL), intraventricular hemorrhage (IVH) of the brain, and ROP are all considered to be precursors or co-morbidities of cerebral palsy. Because of this, an increased risk for any of these disorders (i.e. PVL, IVH, ROP) also increases the risk for cerebral palsy

Glucose is the primary fuel for the central nervous system. Thus, its proper regulation is essential for healthful development of the brain and retina. Episodes of acute dysglycemia, (that is, acute hypoglycemia and/or acute hyperglycemia) in infants has been associated with neurodevelopmental disability. From this, it follows that detecting chronic dysglycemia, that is, dysglycemia that persists for weeks at a time, will give even further insight into whether an infant is at risk of incurring one or more neurodevelopmental disorders. Thus, there is a need for new clinical methodologies that establish the status of chronic glucose regulation, in assessing for the risk of neurodevelopmental disorders. By detecting the presence of chronic hypoglycemia or chronic hyperglycemi—in utero and/or ex utero—appropriate treatment can then be determined and administered.

### SUMMARY

General Definitions: As used herein, ‘neurodevelopmental disorders’ comprise a group of disorders that affect the development of the brain and the retina. Development of the child's brain begins in utero and continues after birth. ‘Neurodevelopmental disorders’ encompass intellectual or cognitive disabilities (such as brain injury arising from intraventricular hemorrhage), behavioral disorders (such as attention deficit hyperactivity disorder), autism spectrum disorders (such as autism), language or learning disorders (such as dyslexia), motor disorders (such as cerebral palsy), neuroretinal and visual disorders (such as retinopathy of prematurity), etc.

As used herein, the infant eye disease known as ‘retinopathy of prematurity’ will be referred to as ‘ROP’. ROP has mild and severe forms. Herein, the terms ‘mild ROP’, ‘early stage ROP’, ‘ROP stage 1 or stage 2’, or ‘non-proliferative ROP’ or ‘hypoglycemic ROP’ are used interchangeably. Herein, the terms severe ROP, late stage ROP, proliferative ROP, and/or ROP stage 3, stage 4, or stage 5 or ‘DD-ROP’ are used interchangeably.

As used herein, the term ‘infant’ means a baby from birth to the second birthday. The term ‘infant’ encompasses ‘preterm infant,’ which means a baby born before 37 completed weeks of gestation. In more severe cases, the preterm infant is born at less than 30 weeks of gestational age or has a birthweight under 1.5 kg. As used herein, ‘developmental diabetes’ (or ‘DD’) refers to a clinical entity that is not yet recognized by the medical community. I will define developmental diabetes as a condition occurring in an infant—most commonly in a preterm infant—which is characterized by chronic hyperglycemia (i.e. hyperglycemia of more than 2 week duration) with an onset before 3 months of chronologic age. Herein, the terms ‘developmental diabetes’ and ‘DD’ will be used interchangeably. The hyperglycemia of DD may have originated before or after birth.

I will define the hallmark of untreated DD, when it occurs in premature infants, as the development of proliferative ROP. In other words, I define proliferative ROP as a complication of untreated DD. Intraventricular hemorrhage (also called “IVH”) of the brain, is another example of a disorder that is mediated by a diabetic-like, microvascular pathology and has been associated with neonatal hyperglycemia. Although IVH is more common in premature infants, it also occurs in full term infants. Thus, IVH is another potential complication of untreated DD—and can occur in any infant (i.e. full term or preterm) with DD.

Of note, DD is a non-genetic condition, which is distinct from neonatal diabetes, which is a hereditary, genetic condition (arising from known mutations). DD is also a different disorder than gestational diabetes because DD pertains to the fetus or infant, whereas gestational diabetes pertains to the mother. That said, DD (of the fetus) and gestational diabetes (of the mother) will overlap, to some extent, during pregnancy. Of note, the diagnosis of gestational diabetes is currently made via an oral glucose tolerance test, and not via a glucose control indicator. In fact, the most conventional glucose control indicator, that is, the standard Hemoglobin A1C assay, is not validated for use in infants or in pregnant women.

The methods of my invention rely upon analysis of a blood sample. As used herein, ‘blood sample’ means a sample of whole blood, or fraction there from (such as serum or plasma), collected from a person (or animal). For example, purple or lavender-topped vacutainer blood collection tubes are used when liquid whole blood is needed for analysis, such as in complete blood counts (CBC). These purple/lavender tubes contain EDTA to prevent clotting. There are other techniques for obtaining plasma or serum specimens, as conventionally known in the art.

The blood sample may be provided in any suitable form, including liquid or dried blood. The blood sample may be freshly-drawn or may be stored. For example, in obtaining dried blood spots, the collected blood is spotted onto filter paper, dried, and then stored for further processing later. The content of the dried blood spot could be eluted into a liquid fluid to perform the analytic measurements. This technique is described in Nico Gruner et al, “Dried Blood Spots—Preparing and Processing for Use in Immunoassays and in Molecular Techniques” (2015) J Vis Exp. 97:52619. The blood could be drawn from the patient using any suitable technique, such as by venipuncture or skin puncture for capillary blood (e.g. finger stick) as commonly used in point-of-care machines.

The methods of my invention rely on performing an analysis of the blood sample for an indicator of extended duration (e.g. intermediate or long-term) of glucose control. My methodology does not involve the direct measurement of glucose levels. Instead, my methodology involves establishing the composite status of glucose control for a duration of at least two weeks (hereinafter ‘glucose control indicator’). In other words, glucose control indicators establish the patient's glucose status as: normal (euglycemia), chronically high (i.e. chronic hyperglycemia) or chronically low (i.e. chronic hypoglycemia). Glucose control indicators also provide information about the severity of dysglycemia (for example, the threshold severity level for designating chronic dysglycemia is 50% above/below the mean). In the case of a fetus or newborn infant, the glucose status may represent the in utero status, the ex utero status, or a combination of the two.

Examples of ‘glucose control indicators’ include the amounts of glycated blood proteins (such as glycated hemoglobin or glycated albumin) in the blood sample. Other examples of ‘glucose control indicators’ include fructosamine and 1,5-anhydroglucitol (1,5-AG). The most conventional ‘glucose control indicator’ is the standard hemoglobin A1c test, which is the percentage fraction of glycated hemoglobin relative to total hemoglobin.

As used herein, ‘glycated hemoglobin’ encompasses any form of hemoglobin that is present in the blood sample, whether adult form (i.e. hemoglobin A and hemoglobin A2) or fetal form (hemoglobin F). The amount of glycated hemoglobin could be detected using any analytic technique, including any conventional technique such as boronate affinity chromatography, immunoassay, high-performance liquid chromatography (HPLC), or ion-exchange chromatography. When glycated albumin is measured, it could also be detected using any analytic technique, including any conventional technique such as boronate affinity chromatography, ion exchange chromatography, thiobarbituric acid assay, immunoassay, enzyme-linked boronate-immunoassay, enzymatic assays, and high-performance liquid chromatography.

These glucose control indicators may be represented in useful form as fractions thereof i.e. ‘glycated hemoglobin fraction’ or ‘glycated albumin fraction.’ These fractions could be expressed in various numeric formats, including percentages or X:Y ratios. The proportion could be expressed as glycated hemoglobin (or albumin) relative to ‘total protein,’ or vice versa. This proportion is calculated using the same units of measure typical for measuring protein amounts (e.g. on a weight-to-weight or gram-to-gram basis).

The term ‘total protein’ is a generic term that encompasses the various possible ways of measuring blood proteins for glucose control indicators. Detecting protein for measuring the amount of ‘total protein’ could be performed by any protein analytic technique, including any conventional technique such as Biuret techniques (such as BCA or Lowry assays), colorimetric dye-based techniques (Bradford stain), fluorescent dye techniques, or UV absorption techniques.

Currently, glycated hemoglobin (or glycated albumin) fractions are calculated relative to total hemoglobin (or total albumin) in the blood sample. In this particular convention, the term ‘total protein’ refers to the total amount of a single type of blood protein, i.e. ‘total hemoglobin protein’ (or ‘total albumin protein’). In other words, the glycated hemoglobin fraction (or glycated albumin) is conventionally expressed relative to total hemoglobin (or total albumin). Total hemoglobin contains both adult and fetal forms of hemoglobin. The current convention (with total hemoglobin as the denominator) confines the use of glycated hemoglobin assays to populations with unchanging adult/fetal hemoglobin ratios. For this reason, current glycated hemoglobin assays are not used during infancy. For similar reasons, they are also generally not used during pregnancy or post-partum period.

Alternatively, glycated hemoglobin (or glycated albumin) fractions could be calculated relative to different variants of ‘total protein’ in the blood sample. When measuring ‘total protein’ (in any of its variants described below), both the glycated hemoglobin (or albumin) and the ‘total protein’ could be measured from the same blood sample (i.e. not from a different tube/vial, or different blood draw collection, or different dried blood spot). For each of these variants of ‘total protein,’ there may be different protocols for collecting the appropriate blood sample or processing thereof, such as special collections tubes or centrifuging.

As used herein, ‘total serum protein’ means the protein contained in the serum portion of blood. Typically, this is measured by collecting blood in a gold or red-top tube (clot activator) which is made to clot and centrifuged to separate out the blood cells. As such, total serum protein contains soluble proteins such as albumins and globulins, but does not contain the proteins involved in blood clotting (e.g. fibrinogen), and also does not contain the protein inside cells.

As used herein, ‘total plasma protein’ means the protein detected in the plasma, which includes the serum portion of blood, plus the clotting factor proteins (e.g. fibrinogen), but not proteins inside blood cells. As such, ‘total plasma protein’ contains the proteins in serum, plus the protein involved in blood clotting (e.g. fibrinogen), but does not contain the protein inside cells. Typically, this is measured from blood collected in a blue-top tube (sodium citrate), which does not cause clotting.

As used herein, ‘total whole blood protein’ means all the proteins in whole blood, which includes all the protein in plasma (and serum) and blood cells. As such, this further includes protein contained within or carried by blood cells (e.g. white and red blood cells). My invention incorporates the measurement of total whole blood protein into a new methodology for assessing chronic dysglycemia. Moreover, my invention defines an original technique for calculating glycated hemoglobin fraction or glycated albumin fraction. I call this novel measurement ‘comprehensive glycated hemoglobin fraction’ (or ‘comprehensive glycated albumin fraction’). More details about this are given below.

Reference Levels: My invention may use reference levels that are currently in use for the relevant glucose control indicators. Alternatively, my invention also considers the possibility that newly developed reference levels for the glucose control indicators may be more applicable for certain populations. These new reference would be different from those that are conventionally used. The reference levels could be specific for premature infants and different from those for full-term infants, children, and adults. The reference levels could also be specific to pregnant women or post-partum women. Also, the reference levels could vary according to the gestational duration and chronological age of the preterm infants. These reference levels may be determined by further clinical studies that explore typical test values found in premature infants, pregnant women, or post-partum women. These newly developed reference levels may differ by the medical condition being assessed.

Aspect #1: Neurodevelopmental Disorder & Developmental Diabetes In one aspect, my invention is a new use for glucose control indicators for assessing a person's risk of incurring a neurodevelopmental disorder or developmental diabetes (also referred to as ‘DD’).This risk assessment could be performed while the person is in-utero (fetus) or live born. The method is performed on a person's blood sample. In the case that the person is a fetus, this means the maternal blood being used as a surrogate of fetal blood. In the case that the person is a newborn infant, this could mean blood taken from the infant or placenta or umbilical cord. The method further comprises utilizing an appropriate glucose control indicator as described elsewhere herein. Any of such various reference levels that define chronic dysglycemia (hyperglycemia or hypoglycemia), as described elsewhere herein, may be used. The measured glucose control indicator could be compared against one or more reference levels as described in more detail elsewhere herein.

Neurodevelopmental Disorder. For the risk assessment of neurodevelopmental disorders, appropriate glucose control indicators that are above a reference level (indicating chronic hyperglycemia) or below a reference level (indicating chronic hypoglycemia) would indicate a risk of incurring one or more neurodevelopmental disorders. With testing on a series of blood samples (i.e. repeat samples taken at different times) from the same person, my invention can be used to monitor the person's risk of acquiring neurodevelopmental disorders over time.

One example in particular is the reference levels for ROP using the ‘comprehensive glycated hemoglobin fraction’ (which is described in more detail herein; see Aspect #2 below). This particular set of reference levels has been actually studied clinically, as described in the Experimental Work section below. These may be representative of reference levels that define the risk for neurodevelopmental disorders, in particular for ROP (though not limited to this disorder). In this particular embodiment, one of the reference levels is a predefined threshold value that is 149% or more (e.g. 150% or 175%) relative to the mean value of that in premature infants without ROP. A test result above that threshold value is indicative of increased risk of incurring a neurodevelopmental disorder (which is associated with hyperglycemia), such as proliferative ROP, and could be treated therefore accordingly. Another of the reference levels is a predefined threshold value that is 71% or less (e.g. 65% or 55%) relative to the mean value of that in premature infants without ROP. A test result below that threshold value is indicative of increased risk of incurring a neurodevelopmental disorder (which is associated with chronic hypoglycemia) such as non-proliferative ROP—and could be treated therefore accordingly.

Developmental Diabetes (DD)., Herein, the term “risk of incurring DD” will be used interchangeably with “the risk of incurring the complications of untreated DD”. One aspect of my invention is the use of a glucose control indicator to determine risk of incurring DD. In one embodiment, my ‘comprehensive glycated hemoglobin (or glycated albumin) fraction’ (described below under Aspect #2) can be utilized for this purpose. When the glycated hemoglobin fraction exceeds an appropriate reference level, the risk of incurring DD are increased.

The use of glucose control indicators for risk assessment of fetal DD is helpful even when the mother has not been diagnosed with gestational diabetes (a condition which is diagnosed via oral glucose tolerance test). The methods of this invention can be performed earlier in pregnancy than the oral glucose tolerance test. Therefore, in some cases, the mother may be susceptible to gestational diabetes but has not yet have been assessed for it. Thus, the methods of this invention can lead to earlier treatment and better health outcomes for both the mother and the developing fetus. With serial testing, my invention can be used to monitor the child's risk of DD over the course of the pregnancy. Of important note, the conventional hemoglobin A1c test is not valid for use during pregnancy.

Treatment. In some embodiments, the method further comprises treating the person based on the risk of incurring a neurodevelopmental disorder or developmental diabetes. In premature infants and newborn infants of diabetic mothers, acute hypoglycemia (diagnosed by actual blood glucose level) is a more common occurrence than acute hyperglycemia. Because of this, premature infants and newborn infants of diabetic mothers when hospitalized, often receive intravenous glucose solutions to boost their blood glucose levels. The glucose solutions are usually administered, knowing the acute glucose status, but without knowledge of the chronic glucose status of the infant. The two common choices for fluid administration are D5W (dextrose 5% in water) and D10W (dextrose 10% in water). Of note, dextrose is a crystalline form of glucose and is a type of sugar that raises blood glucose. Herein, in reference to glucose supplementation (whether oral, enteral, parenteral, topical, or by any other route of administration), the terms “glucose” and “dextrose” will be used interchangeably.

For this particular embodiment, the treatment is made in the setting where the infant is receiving intravenous fluid containing dextrose for fluid management. The treatment can be adjusted or changed to help normalize the infant's glucose trajectory. In cases where the glucose control indicator indicates chronic hyperglycemia, the treatment would be to change to a different intravenous fluid containing a lower concentration of dextrose (e.g. switching from D10W to D5W (D5W), or eliminating dextrose from the intravenous fluid, or reducing the rate of fluid infusion containing the dextrose,) or shortening the duration of the dextrose fluid infusion. In some cases, the infant is treated with a glucose lowering medication (e.g. insulin). In cases where the glucose control indicator indicates chronic hypoglycemia, the treatment would be to change to a different intravenous fluid containing a higher concentration of dextrose (e.g. switching from D5W to D10W), or increasing the rate of fluid infusion containing the dextrose, or prolonging the duration of dextrose fluid infusion. Another treatment for chronic hypoglycemia in infants is the use of a neonatal dextrose gel. In cases of chronic hypoglycemia, the infant's glucose levels can be enhanced with the use of such a gel.

In the particular case of assessing for risk of DD in an infant (with or without maternal gestational diabetes) treatment may be applied to the mother. If the results indicate risk of DD in the to-be-born infant, then a treatment for the mother may be selected or modified. For example, the mother may begin treatment with a glucose lowering medication (e.g. insulin) or have the dosage of such medicine increased. Or the mother may be prescribed an appropriate diet and exercise regimen with repeat risk assessments of DD to monitor the condition.

In this invention, in situations where circumstances warrant, any suitable treatment for hyperglycemia or reducing glucose levels may be used. Examples include initiating a glucose lowering medication (e.g. insulin or oral antihyperglycemic agents), increasing the dosage thereof, or making recommendations for an appropriate diet and exercise regimen. Other examples include modifying, reducing, or eliminating any form of glucose nourishment that the person is receiving . Examples of glucose nourishment include intravenous fluids containing dextrose or glucose supplementation by any route of administration, such as oral (e.g. baby formula), enteral, parenteral, or topical. In situations where circumstances warrant, any suitable treatment for hypoglycemia or raising glucose levels may be used. Examples include initiating administration of glucose nourishment or increasing the amount thereof (e.g. changing to intravenous fluid containing a higher dextrose concentration or administering a glucose supplement by oral, enteral, parenteral, topical, or any other route of administration.)

Aspect #2: Comprehensive Glycated Hemoglobin (or Glycated Albumin) Fraction: In another aspect, my invention is a novel method for evaluating the amount of glycated hemoglobin (or glycated albumin) in a blood sample. My technique which I call ‘comprehensive glycated hemoglobin (or glycated albumin) fraction’ requires the measurements of both glycated hemoglobin (or glycated albumin) and ‘total whole blood protein’. Both the glycated hemoglobin (or glycated albumin) and the ‘total whole blood protein’ are measured from the same blood sample (i.e. not from a different tube/vial, or different blood draw collection, or different dried blood spot). As explained above, the term ‘total whole blood protein’ means all the proteins in whole blood, which includes all the protein in plasma (and serum) and blood cells (e.g. red and white blood cells). To access the proteins inside blood cells for measurement of the total whole blood protein, the assay procedure may require lysis of the blood cells, e.g. using cell lysis buffer to break open the cells so that the proteins are released into the liquid sample or so that the protein-measuring reagents have access to the intracellular proteins. The protein measurement could be performed after the cell lysis of the blood sample.

Conventionally, glycated hemoglobin is expressed as a ratio relative to total hemoglobin in blood. In my invention, my ‘comprehensive glycated hemoglobin (or glycated albumin) fraction’ is obtained by calculating the amount of glycated hemoglobin (or glycated albumin) relative to ‘total whole blood protein’. This proportion could be expressed in various numeric formats, including percentages or X:Y ratios. This proportion is calculated using the same units of measure typical for measuring protein amounts (e.g. on a weight-to-weight or gram-to-gram basis). For example, ‘comprehensive glycated hemoglobin fraction’ could be expressed as [glycated hemoglobin]/['total whole blood protein']%. And similarly for ‘comprehensive glycated albumin fraction.’

The methods of my invention are a more effective way of considering glycated hemoglobin (or glycated albumin) as a relevant glucose control biomarker in any person. However, it is particularly useful when the standard hemoglobin A1c methodology is not accurate. This is especially true during pregnancy, infancy and post-partum when the adult hemoglobin to fetal hemoglobin ratio is variable. My comprehensive glycated hemoglobin fraction is more accurate in these situations because total hemoglobin (made up of adult and fetal hemoglobin) is just one component of total whole blood proteins. Therefore, my methodology is not significantly impacted by the adult/fetal hemoglobin ratio.

Aspect #3: Disorders of Glucose Metabolism. In another aspect, my invention is a method for using the calculated ‘comprehensive glycated hemoglobin (or glycated albumin) fraction’ (which is described above). My inventive method can be used to diagnose, assess risk, or establish a treatment plan for medical conditions characterized by dysfunctional glucose metabolism. One such group of disorders comprises the various types of diabetes conditions, including type 1 diabetes, type 2 diabetes, gestational diabetes, neonatal diabetes and DD (described elsewhere herein). It can also be used in other types of dysfunctional glucose metabolism conditions including insulin resistance, metabolic syndrome, postpartum hyperglycemia in gestational diabetics and chronic infant hypoglycemia (i.e. of at least 14 days duration). My methodology could be used in any relevant patient population, including adults, children, or infants, and also other animals (such as cats and dogs).

The invention method further comprises comparing the ‘comprehensive glycated hemoglobin (or glycated albumin) fraction’ to one or more reference levels. The reference levels may be any of those described herein. When the comprehensive glycated hemoglobin (or glycated albumin) fraction exceeds the relevant reference level, then chronic hyperglycemia is present. The method may further comprise treating the medical condition in any suitable manner, such as administering a glucose lowering medication (such as insulin or oral diabetes drugs) or by conservatively managing the condition (such as with diet and exercise). More details of this have been provided elsewhere herein. When the ‘comprehensive glycated hemoglobin (or glycated albumin) fraction’ is below the relevant reference level, then chronic hypoglycemia is present and treatment to raise glucose levels by glucose supplementation can be carried out (as described elsewhere herein).

My invention methodology could also be performed on sequential samples (repeat testing at multiple different times), drawn from the same person, for the purposes of monitoring any of the aforementioned medical conditions. For example, the results from an initial blood sample (or at least, one of the person's blood samples), could be used to make the diagnosis of the medical condition, establish a baseline value for the glucose control indicator, or determine a treatment plan. Subsequent to that, results from samples, drawn at different times, can be compared to one another. This allows for the medical condition to be monitored and the person's treatment to be adjusted as necessary.

### BRIEF DESCRIPTION OF THE DRAWINGS

FIG. 1 shows the results of the preterm infant study that was performed.

### DETAILED DESCRIPTION OF EXAMPLES EMBODIMENT

To assist in understanding the invention, the following example embodiments are described in more detail. Example Procedure: Collect blood from the patient (which could also be maternal, placental, or umbilical cord blood, as explained above). The blood can be either liquid blood or blood dried on filter paper (which is a dried blood spot). If the blood is dried on filter paper, elute the blood from the filter paper and perform measurements on the eluted fluid. Lyse the red blood cells in the blood sample using any suitable technique, such as immersing in lysis buffer. Measure the amount of glycated hemoglobin using any conventional technique, such as high-pressure liquid chromatography, boronate affinity, or immunoassay. Measure the amount of total protein in the cell-lysed blood sample using any conventional technique, such as by Biuret reagent, Kjeldahl method, dye-binding, or refractometry. One particular example is the “Micro BCA Protein Assay Kit” sold by various laboratory suppliers. Use appropriate statistical analysis to interpret the results. Because the results represent approximately one month (prior) of blood glucose control levels, repeat the testing at multiple different times, such as at or near birth (cord blood or within a week of birth), one week old, four weeks old, eight weeks old, 12 weeks old, etc.

Experimental Work: This was a cross-sectional study of 43 preterm infants born at 26-28 weeks gestational age. For each infant, blood samples were drawn after birth at <1 week chronological age, and again at approximately 4 weeks of age. These blood samples were stored as dried blood spots (DBS) for further analysis. In preparation for measuring glycated hemoglobin (HbA1c), the DBS were soaked in 120 μl of RBC lysis buffer for 60 min at room temperature. Samples were centrifuged at 13,000 rpm for 5 min at 4° C. Supernatants were collected and stored on ice. The resulting liquid samples were diluted 1:4000 with sample diluent (supplied with kit).

Glycated hemoglobin (HbA1c) in the liquid samples (DBS extracts) were measured by ELISA. 50 μl of standard and samples, and 50 μl of biotin-conjugate solution (except blank wells) were put in ELISA plate wells. Plates were sealed and incubated for 40 min at 37° C. Plate wells were decanted and washed with wash buffer. 100 μl of HRP-conjugate solution was then added to all the wells (except blank wells). The plates were sealed and incubated for 40 min at 37° C. The plate wells were washed with wash buffer. 90 μl of substrate solution added to each well. The plates were sealed and incubated for 20 min at 37° C. in dark. To stop the color development, 50 μl of stop solution was added to each well. The plate was immediately read with 450 nm light.

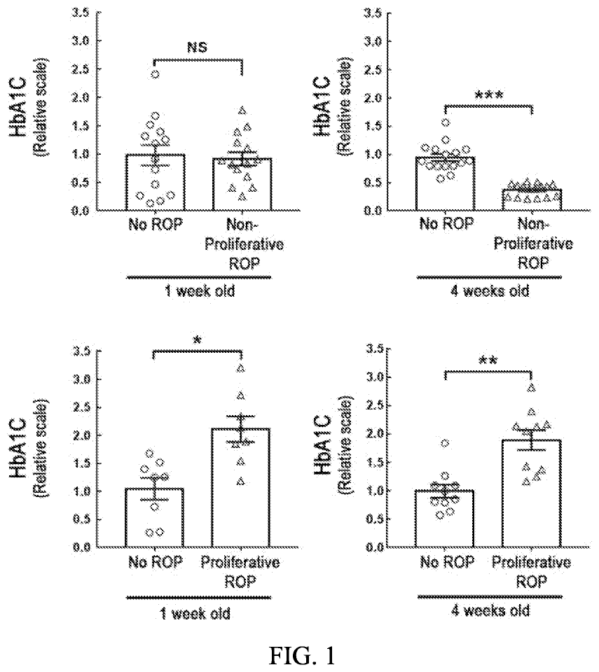

This study sought to find correlations with the relative amount of HbA1c expressed as concentration in relation to the relative amount of total whole blood protein in the DBS extract. Towards this objective, the total whole blood protein (mg) contained in the DBS extracts were measured using a BCA protein assay kit. FIG. 1 shows the results of this study. The bar graphs indicate the relative hemoglobin A1c levels in the various groups. To produce the bar graphs, the data from each assay were normalized to the mean of the matched controls (i.e. premature infants with no ROP). In other words, the controls were set to 1.0 on a relative HbA1c scale. Non-parametric matched t-tests were utilized for statistical comparisons between matched pairs. Each graph represents pairs of infants (no ROP vs non-proliferative ROP seen in top graphs; no ROP versus proliferative ROP seen in bottom graphs) matched by sex and birthweight. NS=non-significant; *p<0.05, **p<0.01, ***p<0.001.

In addition to humans, this invention could also be used in other mammalian animals as well. The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

Hypothetical Insights: I propose various insights about the relationship between blood glucose, diabetes, and ROP. However, these hypotheses are for informative and academic purposes only and are not intended to limit the invention in any way. I teach that ROP is not one disease with two phases (as currently believed). Instead, I teach that ROP, like cancer, is actually multiple diseases. I teach that a novel disorder that I will call ‘hypoglycemic ROP’ is one type of ROP disease. I teach that the cases fitting the hypoglycemic ROP disease category are those that are currently classified as mild ROP, ROP Stage 1 or Stage 2 or non-proliferative ROP (NP-ROP) which regress (and do not progress to the proliferative stages).

I teach that ‘hypoglycemic ROP’ can be a clinical marker of hypoglycemia in preterm infants to establish reference levels for chronic hypoglycemia (of about a month's duration) in preterm infants as part of a metric to estimate glucose control. It is because of the lack of any clinical cut-off points to establish criteria for hypoglycemia that the hemoglobin A1c test (and other glucose control metrics) are not currently used to monitor chronic hypoglycemia.

I teach that it is faulty methodology in glucose evaluation that has previously led to the difficulty in establishing the link between hypoglycemia and neurodevelopmental impairment (including neuroretinal/visual disability). In prior studies, a glucose control indicator (such as described in my invention) has not been used to evaluate the association between hypoglycemia and neurodevelopment. Instead, the prior studies measured the number of hypoglycemic episodes (defined by adult reference levels for hypoglycemia) or average glucose levels. But these do not give an accurate representation of chronic glucose control.

I teach that there is an unrecognized type of diabetes that I designate as ‘developmental diabetes (DD).’ (I define ‘DD’ earlier in this patent application). Like hyperglycemia, DD is characterized by blood glucose levels that are higher than normal for gestational age. I teach that, similar to other types of diabetes, DD causes a host of diabetic-related complications affecting multiple organs (e.g. kidney, eye, brain). In other words, I teach that multiple morbidities of prematurity (such as ROP, intraventricular hemorrhage (IVH), necrotizing enterocolitis, kidney disorders, etc.) should be re-defined as diabetic complications of DD. These morbidities can be prevented by treating DD.

I teach that a disorder that I designate as ‘DD-ROP’ is a type of ROP disease. In this patent application, I refer to DD-ROP by its more traditional names “proliferative ROP” or “Late Stage ROP). I teach that DD-ROP can be used as a clinical marker of DD in preterm infants to establish reference levels for chronic hyperglycemia associated with DD, in preterm infants as part of a metric to estimate glucose control. It can be used in any methodology for assessing glucose control, such as the described in this patent application or in other glucose control metrics. I teach that the cases fitting the DD-ROP disease category type are those that are currently classified as proliferative ROP. I teach that DD-ROP can be used in the methodology for assessing glucose control described in this patent application. I teach that my glycated hemoglobin measurement technique can be used to estimate the risk of short term (within the first 6 months of life) and long-term complications from DD. These complications include neuro-retinal diseases (e.g. proliferative ROP) as well as various types of neurodevelopmental impairment.

I teach that it is faulty methodology in glucose evaluation that has previously led to the difficulty in establishing the link between hyperglycemia and proliferative ROP. In previous studies, chronic glucose control indicators were not used to evaluate the association between ROP and chronic hyperglycemia. Instead, they measured the number of hyperglycemic episodes (defined by adult reference levels for hyperglycemia) or average glucose levels. Because these are surrogate measures of hyperglycemia, they do not give an accurate representation of chronic glucose control.

1. A method for assessing the risk of a neurodevelopmental disorder in a person, comprising:
having a blood sample of the person;
measuring a glucose control indicator in the blood sample;
comparing the measured glucose control indicator to a reference level;
based on the comparison, determining the risk of developing a neurodevelopmental disorder.

2. The method of claim 1, wherein the glucose control indicator is a glycated hemoglobin fraction;
wherein the step of measuring the glucose control indicator comprises assaying the blood sample for glycated hemoglobin and total protein;
wherein the glycated hemoglobin fraction is calculated as the amount of glycated hemoglobin relative to total protein.

3. The method of claim 2, wherein the glucose control indicator is comprehensive glycated hemoglobin fraction; wherein the total protein is total whole blood protein.

4. The method of claim 1, wherein the glucose control indicator is glycated albumin fraction;
wherein the step of measuring the glucose control indicator comprises assaying the blood sample for glycated albumin and total protein;
wherein the glycated hemoglobin fraction is calculated as the amount of glycated albumin relative to the total protein.

5. The method of claim 4, wherein the glucose control indicator is comprehensive glycated albumin fraction; wherein the total protein is total whole blood protein.

6. The method of claim 1, wherein the person is a preterm infant and the neurodevelopmental disorder is retinopathy of prematurity.

7. The method of claim 6, wherein the glucose control indicator being below the reference level indicates a risk of developing non-proliferative retinopathy of prematurity.

8. The method of claim 6, wherein the glucose control indicator being above the reference level indicates a risk of developing proliferative retinopathy of prematurity.

9. The method of claim 1, wherein the person is an infant and the neurodevelopmental disorder is intraventricular hemorrhage.

10. The method of claim 1, wherein the person is a fetus and the blood sample is obtained from the mother.

11. The method of claim 1, wherein the blood sample is provided as a dried blood spot.

12. The method of claim 11, further comprising eluting the dried blood spot into a liquid fluid, wherein measurement of the glucose control indicator is performed on the eluted fluid; and
further comprising measuring total protein in the eluted fluid.

13. The method of claim 1, wherein the person is an infant, and further comprising having multiple blood samples obtained from different times;
wherein at least one blood sample is obtained before birth, at birth, within a week of birth, or at about one week age;
wherein at least another blood sample is obtained at about four weeks age or after;
wherein the glucose control indicator is measured from each one of the multiple blood samples.

14. The method of claim 1, wherein the method is further for treating the person at risk of developing a neurodevelopmental disorder, and the method further comprises:
in the situation where the glucose control indicator is above the reference level, performing the step of: (i) initiating a glucose lowering medication, (ii) increasing the dosage thereof, or (iii) reducing or eliminating or modifying any glucose supplementation that the person is receiving;
in the situation where the glucose control indicator is below the reference level, performing the step of: (i) initiating administration of a glucose or dextrose nourishment, or (ii) increasing the amount thereof.

15. A method of determining a glucose control status in a person, comprising:
having a blood sample of the person;
measuring the amount of glycated hemoglobin or glycated albumin in the blood sample;
measuring the amount of total whole blood protein in the blood sample;
calculating a comprehensive glycated hemoglobin fraction or a comprehensive glycated albumin fraction as the amount of glycated hemoglobin or amount of glycated albumin, respectively, relative to total whole blood protein;
comparing the comprehensive glycated hemoglobin fraction or the comprehensive glycated albumin fraction to a reference level;
based on the comparison, determining the chronic glucose control status of the patient.

16. The method of claim 15, wherein the glucose control indicator being above the reference level indicates a diagnosis of chronic hyperglycemia; and
wherein the method is further for treating the person for chronic hyperglycemia by administering a glucose lowering medication or with exercise and diet.

17. The method of claim 15, wherein the person is a pregnant or post-partum woman and the chronic hyperglycemia is from gestational diabetes.

18. The method of claim 15, further comprising having multiple blood samples obtained from different times;
wherein at least one blood sample is used to make a diagnosis of a dysglycemic medical condition or establish a baseline value for the glucose control indicator;
wherein a subsequent blood sample is obtained at least four weeks later;
wherein the comprehensive glycated hemoglobin fraction or the comprehensive glycated albumin fraction is calculated as results from each one of the multiple blood samples;
and optionally, wherein the method further comprises (i) using the at least one blood sample to determine a treatment plan for the dysglycemic medical condition; and (ii) comparing the results to one another to monitor and adjust the treatment plan.

19. A method for assessing the risk of developmental diabetes in a fetus or infant, comprising:
having a blood sample;
measuring a glucose control indicator in the blood sample;
comparing the measured glucose control indicator to a reference level;
wherein the glucose control indicator being above the reference level indicates a risk of developing developmental diabetes

20. The method of claim 19, wherein the method is further for treating the fetus or infant at risk of developing a neurodevelopmental disorder; and
the method further comprises performing the step of, on the mother or infant: (i) initiating a glucose lowering medication, (ii) increasing the dosage thereof, or (iii) reducing or eliminating any glucose supplementation.
